(19) United States
(12) Patent Application Publication
(10) Pub. No.: US 2024/0415382 A1
(43) Pub. Date: Dec. 19, 2024

(54) DISPOSABLE BLADE LARYNGOSCOPE AND PROCESSING METHOD THEREOF

(71) Applicant: Zhuhai Weishi Medical Technology Co., Ltd., Zhuhai (CN)

(72) Inventors: Qibiao XIONG, Zhuhai (CN); Jiacheng CHEN, Zhuhai (CN); Lianhao QIU, Zhuhai (CN); Kunpeng ZHANG, Zhuhai (CN); Lang LIU, Zhuhai (CN); Ruiliang XIE, Zhuhai (CN)

(73) Assignee: Zhuhai Weishi Medical Technology Co., Ltd., Zhuhai (CN)

(21) Appl. No.: 18/657,476

(22) Filed: May 7, 2024

(30) Foreign Application Priority Data
Jun. 19, 2023 (CN) 202310729658.5

(51) Int. Cl. A61B 1/267; A61B 1/00

(52) U.S. Cl. CPC A61B 1/267; A61B 1/00018; A61B 1/00103; A61B 1/0011; A61B 1/00128

(57) ABSTRACT

Disclosed a disposable blade laryngoscope and a processing method thereof. For the laryngoscope of the present disclosure, a first shell and a second shell are designed on the basis of an original laryngoscope, which is beneficial for installing an optical lens assembly, and meanwhile, a blade may serve as a protection shell of the optical lens assembly, whereby the optical lens assembly may be supported in a use process to be prevented from being pressed to deform by the epiglottis of the human body in a use process. In addition, one layer of coating shell is formed on the blade by secondary injection molding, whereby the first shell and the second shell may be effectively sealed and fixed, the installation process may be reduced, and the assembly efficiency may be improved; and meanwhile, a tongue depressor and a handle may be integrally formed.

### CROSS-REFERENCE TO RELATED APPLICATIONS

This application claims priority to Chinese Patent Application No. 202310729658.5, filed on Jun. 19, 2023, the content of which is incorporated herein by reference in its entirety.

### TECHNICAL FIELD

The present disclosure herein relates to the technical field of laryngoscopes, and in particular to a disposable blade laryngoscope and a processing method thereof.

### BACKGROUND

A video laryngoscope is a new choice for difficult clinical tracheal intubation in recent years, on one hand, it solves the limitation of visual field of human eyes, on the other hand, its laryngoscope lens conforms to the anatomical angle of human throat, thereby being beneficial to exposure of glottis and reducing the difficulty of tracheal intubation. The video laryngoscope changes the tracheal intubation mode of a traditional laryngoscope, and shifts the sight line from the outside of the mouth to the front end of the laryngoscope, thus becoming a high-efficiency and convenient tool for solving difficult clinical intubation at present, which obtains more and more extensive clinical application.

The video laryngoscope collects images of epiglottis and glottis anatomical structures through an image sensor at a handle, and transmits the images to a display screen through a video transmission line, whereby a doctor observes the whole process of tracheal intubation, and assists in intubation through the handle. The video laryngoscope generally includes a laryngoscope lens, an optical lens, a display and the like, and the laryngoscope lens contacts the oral mucosa of a patient and needs to meet related biocompatibility test requirements, at least including cytotoxicity, oral mucosa stimulation and sensitization reaction.

A common structure of an existing laryngoscope lens on the market at present is that a shell adopts the fixing modes of screw locking, glue bonding, buckle locking and the like, for example, a disposable visual laryngoscope disclosed by Chinese Patent Publication No. CN218279605U, in which two shells are connected by a buckling mode, while the problems of complicated assembly, long consumed time, poor uniformity, high assembly cost, and low volume production exit. Meanwhile, this kind of laryngoscope adopts a plurality of materials, which is not favorable for control of biocompatibility. In addition, due to buckling installation, gaps are inevitably formed between the two shells, the gaps easily cause the body fluid to enter the inside of the laryngoscope to damage visual components, therefore, the laryngoscope needs to be designed with a sealing structure or a unique buckling mechanism to ensure the sealing performance of the shells, and meanwhile, the gaps on the shells easily hide dirt, which is not beneficial to cleaning and protection.

### SUMMARY

In order to overcome one of the defects of the relevant art, an objective of the present disclosure is to provide a disposable blade laryngoscope and a processing method thereof, in which the disposable blade laryngoscope is simple in structure and good in sealing performance, and the processing method is convenient in processing, which are beneficial for improving the sealing performance of the laryngoscope.

In order to solve the above problems, the technical solution adopted by the present disclosure is as follows.

A disposable blade laryngoscope includes a blade, a coating shell and an optical lens assembly, and the blade includes a first shell and a second shell which are buckled to form a mounting channel; one end of the mounting channel has a mounting clamping hole, and the other end forms a mounting fixed seat; the coating shell coats outside the blade, and the end, close to the mounting clamping hole, of the coating shell extends outwards to form a tongue depressor; the end, close to the mounting fixed seat, of the coating shell forms a hollow handle, and the coating shell is provided with a visible hole communicating with the mounting clamping hole; and the optical lens assembly is installed in the mounting channel, the input end of the optical lens assembly is clamped in the mounting clamping hole and penetrates through the coating shell, and the output end of the optical lens assembly is installed on the mounting fixed seat and placed in the handle.

In some possible implementation modes, the optical lens assembly includes a light source module, a camera module and an interface module, the light source module and the camera module are installed in the mounting clamping hole, the light source module and the camera module are connected with the interface module through data lines, the interface module is installed in the mounting fixed seat, and one end of the interface module extends outwards, is placed in the handle and forms a quick insert joint.

In some possible implementation modes, clamping grooves are formed on the inner sides of the ends, close to the mounting clamping hole, of the first shell and the second shell, a transparent window plate is clamped in the clamping groove, and the transparent window plate blocks the mounting clamping hole.

In some possible implementation modes, a plurality of positioning bars are disposed in the first shell and the second shell, and the positioning bars on the first shell and the second shell may be matched with each other to clamp the outer wall of the camera module.

In some possible implementation modes, the first shell and the second shell are connected by ultrasonic welding.

A processing method of a disposable blade laryngoscope includes the following steps:
S100: a first shell and a second shell are obtained by primary injection molding, the first shell and the second shell are buckled to form a blade, the blade has a mounting channel inside, and a mounting clamping hole and a mounting fixed seat are disposed at two ends of the mounting channel;
S200: a light source module, a camera module and an interface module are installed in the mounting channel, the light source module and the camera module are installed in the mounting clamping hole, the interface module is installed on the mounting fixed seat, with the outputting end extending out of the mounting fixed seat, and the light source module and the camera module are connected with the interface module through data lines;
S300: gaps formed after the first shell and the second shell are buckled are sealed by welding through an ultrasonic welding device, thereby obtaining a primary product;
S400: a protection structure is plugged in the mounting clamping hole of the primary product, a protection structure sleeves the part, extending out of the mounting fixed seat, of the interface module, and secondary injection molding of a coating shell is carried out outside the primary product to obtain a product of second injection molding;
S500: the protection structure on the product of second injection molding is detached, and the coating shell is trimmed to obtain the laryngoscope.

In some possible implementation modes, positioning bars are formed at one ends, with the mounting clamping hole, of the first shell and the second shell obtained by primary injection molding in S100 by injection molding; and after the first shell and the second shell are buckled to form the blade, the positioning bars on the first shell and the second shell form part of the structure of the mounting clamping hole.

In some possible implementation modes, in S400, the coating shell of secondary injection molding outside the primary product has a tongue depressor and a hollow handle, the protection structure coating the part, extending out of the mounting fixed seat, of the interface module forms a filler in the injection molding process of the handle, and the production structure plugged in the mounting clamping hole forms a filler of a visible hole in the coating shell.

In some possible implementation modes, in S200, the light source module, the camera module and the interface module are coated with thermal insulation films and installed in the mounting channel together with the thermal insulation films.

In some possible implementation modes, in S200, when the light source module and the camera module are installed in the mounting clamping hole, a transparent window plate needs to be installed at the outward end of the mounting clamping hole.

Compared with the relevant art, the present disclosure has the following beneficial effects.

For the disposable blade laryngoscope of the present disclosure, the first shell and the second shell are designed on the basis of the original laryngoscope, which is beneficial for installing the optical lens assembly, and meanwhile, the blade may serve as a protection shell of the optical lens assembly, whereby the optical lens assembly may be supported in a use process to be prevented from being pressed to deform by the epiglottis of the human body in the use process. In addition, one layer of coating shell is formed on the blade by secondary injection molding, whereby the first shell and the second shell may be effectively sealed and fixed, the installation process may be reduced, and the assembly efficiency may be improved; and meanwhile, the tongue depressor and the handle may be integrally formed, compared with the traditional forming mode of forming the tongue depressor and the handle on the blade, the manufacturing difficulty of a mold is greatly reduced, which is conducive to demolding of the mold and improving the efficiency of processing. For the processing method of the disposable blade laryngoscope, the operation is simple, the assembly is convenient, and the sealing performance of a finished product is good.

### BRIEF DESCRIPTION OF THE DRAWINGS

In order to describe the technical solutions in the embodiments of the present disclosure more clearly, the drawings required to be used in description of the embodiments will be simply introduced below, obviously, the drawings described below are only some embodiments of the present disclosure, and other drawings can further be obtained by those of ordinary skill in the art according to the drawings without creative work.

FIG. 1 is a schematic structure diagram I of an embodiment of the present disclosure.

FIG. 2 is a schematic structure diagram II of an embodiment of the present disclosure.

FIG. 3 is a schematic internal structure diagram of an embodiment of the present disclosure.

FIG. 4 is a schematic structure diagram with a coating shell removed of an embodiment of the present disclosure.

FIG. 5 is a schematic diagram of an exploded view with a coating shell removed of an embodiment of the present disclosure.

FIG. 6 is a processing method of a disposable blade laryngoscope of an embodiment of the present disclosure.

### DETAILED DESCRIPTION OF THE EMBODIMENTS

The technical solutions in the embodiments of the present disclosure are clearly and completely described below in conjunction with the accompanying drawings in the embodiments of the present disclosure. Apparently, the embodiments described are only a part rather than all of the embodiments of the present disclosure. All other embodiments obtained by those of ordinary skill in the art based on the embodiments of the present disclosure without creative efforts shall fall within the protection scope of the present disclosure. In the following, terms “first” and “second” are only adopted for description and should not be understood to indicate or imply relative importance or implicitly indicate the number of indicated technical features. Therefore, a feature defined by “first” and “second” may explicitly or implicitly indicates inclusion of at least one such feature. In the description of the embodiment of the present disclosure, “a plurality of” means two or more, unless otherwise specified.

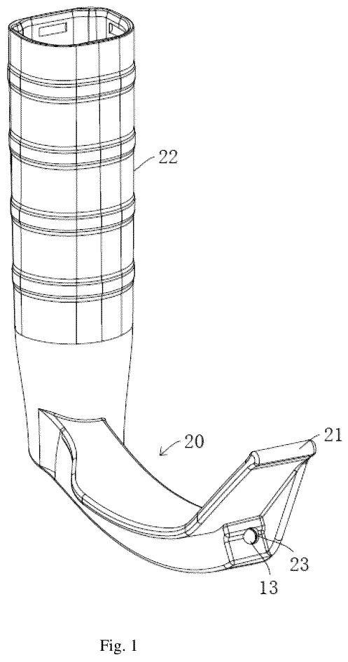

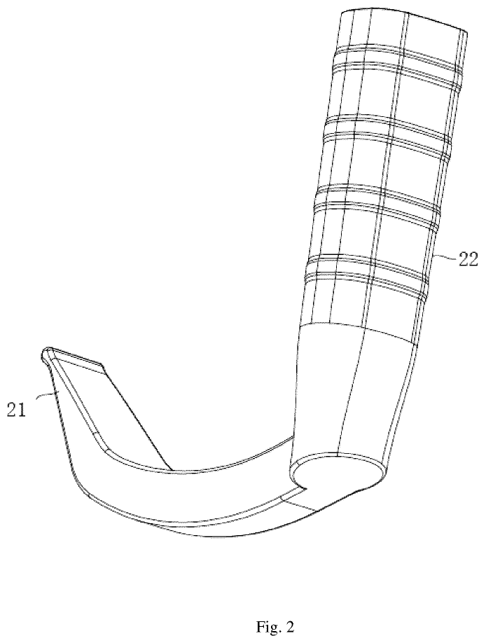

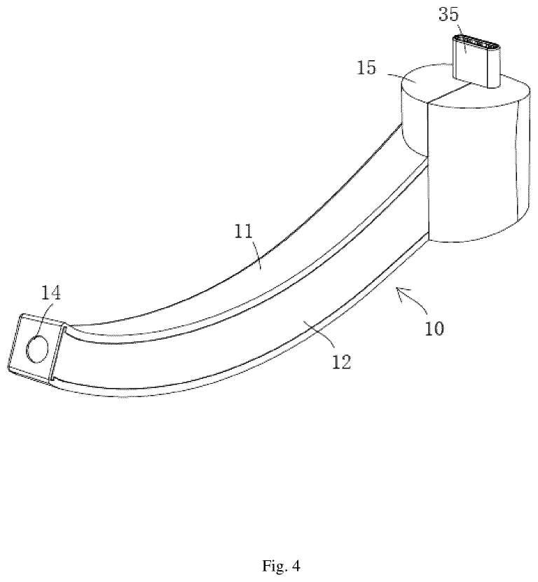

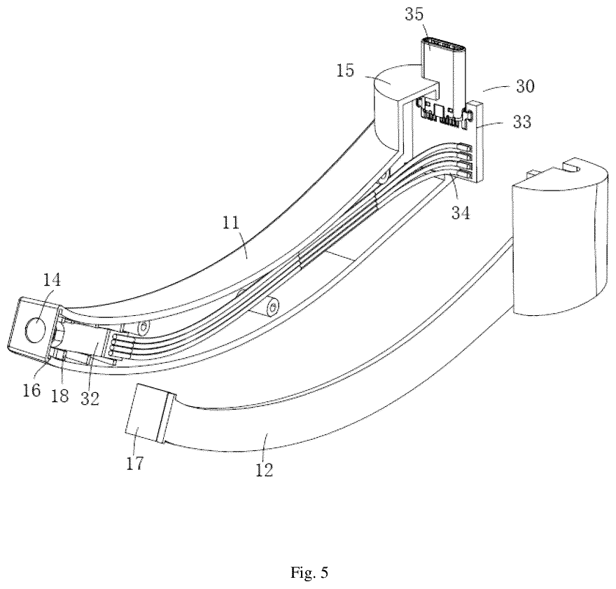

A disposable blade laryngoscope as shown in FIGS. 1-5 includes a blade 10, a coating shell 20 and an optical lens assembly 30, the blade 10 includes a first shell 11 and a second shell 12, and the first shell 11 and a second shell 12 are buckled to form a mounting channel 13; one end of the mounting channel 13 has a mounting clamping hole 14, and the other end forms a mounting fixed seat 15; the coating shell 20 coats outside the blade 10, and the end, close to the mounting clamping hole 14, of the coating shell 20 extends outwards to form a tongue depressor 21; the end, close to the mounting fixed seat 15, of the coating shell 20 forms a hollow handle 22, and the coating shell 20 is provided with a visible hole 23 communicating with the mounting clamping hole 14; and the optical lens assembly 30 is installed in the mounting channel 13, the input end of the optical lens assembly 30 is clamped in the mounting clamping hole 14 and penetrates through the coating shell 20, and the output end of the optical lens assembly 30 is installed on the mounting fixed seat 15 and placed in the handle 22.

The first shell 11 and the second shell 12 are obtained by independent processing in a primary forming mold, at least one of the first shell 11 and the second shell 12 has an inner cavity inside to form the mounting channel 13, whereby a mold for forming the first shell 11 or the second shell 12 needs a mold core to form the mounting channel 13, in such a case, if the tongue depressor 21 and the hollow handle 22 are formed in the mold, the processing difficulty of the corresponding mold and the difficulty of demolding of the mold in the later stage may be increased obviously. While the tongue depressor 21 and the hollow handle 22 do not need to be formed on the mold for forming the first shell 11 and the second shell 12 in the present disclosure, therefore, the mold for forming the first shell 11 and the second shell 12 is simpler, and the production cost may be effectively reduced.

For the disposable blade laryngoscope, the first shell 11 and the second shell 12 are designed on the basis of the original laryngoscope, which is beneficial for installing the optical lens assembly 30, and meanwhile, the blade 10 may serve as a protection shell of the optical lens assembly 30, whereby the optical lens assembly 30 may be supported in a use process to be prevented from being pressed to deform by the epiglottis of the human body in the use process. In addition, one layer of coating shell 20 is formed on the blade 10 by secondary injection molding, whereby the first shell 11 and the second shell 12 may be effectively sealed and fixed, the installation process may be reduced, and the assembly efficiency may be improved; and meanwhile, the tongue depressor 21 and the handle 22 may be integrally formed, compared with the traditional forming mode of forming the tongue depressor 21 and the handle 22 on the blade 10, the manufacturing difficulty of a mold is greatly reduced, which is conducive to demolding of the mold and improving the efficiency of processing.

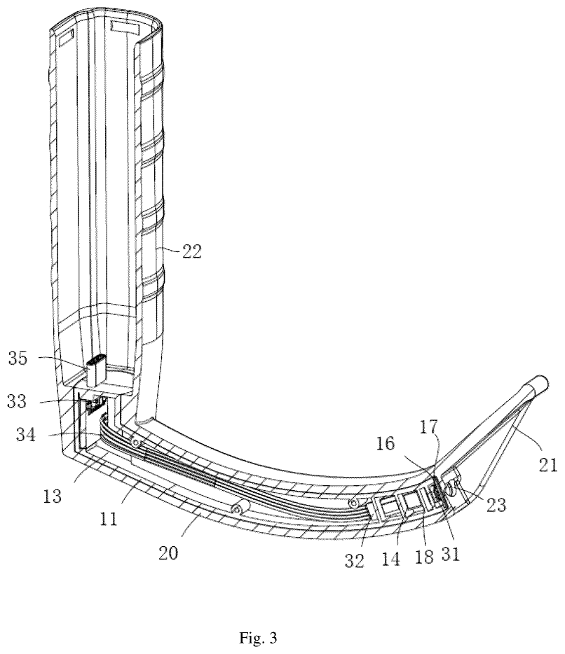

Furthermore, referring to FIGS. 3-5, in some possible implementation modes, the optical lens assembly 30 includes a light source module 31, a camera module 32 and an interface module 33, the light source module 31 and the camera module 32 are installed in the mounting clamping hole 14, the light source module 31 and the camera module 32 are connected with the interface module 33 through data lines 34, the interface module 33 is installed in the mounting fixed seat 15, and one end of the interface module 33 extends outwards, is placed in the handle 22 and forms a quick insert joint 35. The camera module 32 and the light source module 31 are ready-made modules, which will not be elaborated in the present disclosure, and reference can be made to a camera module in a laryngoscope blade disclosed by Chinese Patent Publication No. CN203634128U. During installation, the data line 34 and the interface module 33, the light source module 31 and the camera module 32 are respectively connected by welding, a special welding device or welding jig may be adopted for welding, the operation is relatively simple, and during installation, it only needs to clamp the camera module 32 and the interface module 33 into the mounting clamping hole 14 and the mounting fixed seat 15, the entire installation process is relatively simple.

It needs to be noted that in some cases, the light source module 31 may be integrated with the camera module 32 to simplify the structure, of course, in some embodiments, the light source module 31 and the camera module 32 are separate modules, so the light source module 31 needs to be installed separately on the peripheral side of the mounting clamping hole 14. Of course, in some embodiments, for better installation, the light source module 31 may be designed in a ring shape, and then sleeves the front end of the camera module 32 and is clamped in the mounting clamping hole 14 together.

In some possible implementation modes, in order to facilitate protection of the light source module 31 and the camera module 32 and also improve the sealing performance of the blade 10, clamping grooves 16 are formed on the inner sides of the ends, close to the mounting clamping hole 14, of the first shell 11 and the second shell 12, a transparent window plate 17 is clamped in the clamping groove 16, and the transparent window plate 17 blocks the mounting clamping hole 14. The transparent window plate 17 may protect the light source module 31 and the camera module 32 to a certain degree during secondary injection molding of the coating shell 20.

In some possible implementation modes, in order to improve installation precision of the camera module 32, a plurality of positioning bars 18 are disposed in the first shell 11 and the second shell 12, and the positioning bars 18 on the first shell 11 and the second shell 12 may be matched with each other to clamp the outer wall of the camera module 32. In fact, the positioning bars 18 form part of the structure of the mounting clamping hole 14.

In some possible implementation modes, in order to avoid loose of the first shell 11 and the second shell 12 during the secondary injection molding, and further improving the strength and tightness of the entire blade 10, the first shell 11 and the second shell 12 are connected by ultrasonic welding.

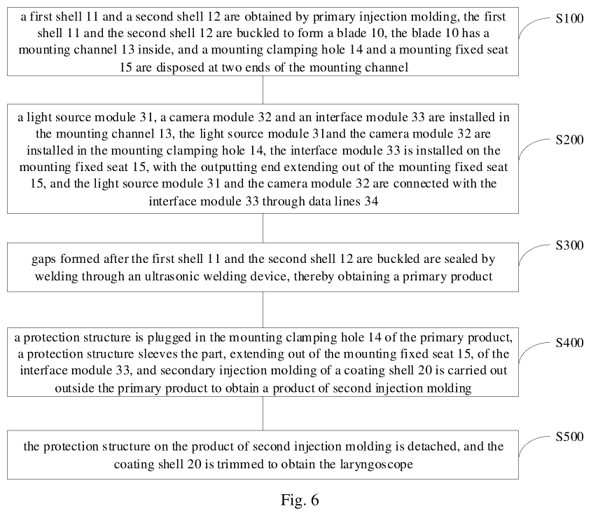

The present disclosure further provides a processing method of a disposable blade laryngoscope. As show in FIG. 6, the processing method is used for processing the disposable blade laryngoscope of the above embodiment, and includes the following steps:
S100: a first shell 11 and a second shell 12 are obtained by primary injection molding, the first shell 11 and the second shell 12 are buckled to form a blade 10, the blade 10 has a mounting channel 13 inside, and a mounting clamping hole 14 and a mounting fixed seat 15 are disposed at two ends of the mounting channel;
S200: a light source module 31, a camera module 32 and an interface module 33 are installed in the mounting channel 13, the light source module 3land the camera module 32 are installed in the mounting clamping hole 14, the interface module 33 is installed on the mounting fixed seat 15, with the outputting end extending out of the mounting fixed seat 15, and the light source module 31 and the camera module 32 are connected with the interface module 33 through data lines 34;
S300: gaps formed after the first shell 11 and the second shell 12 are buckled are sealed by welding through an ultrasonic welding device, thereby obtaining a primary product;
S400: a protection structure is plugged in the mounting clamping hole 14 of the primary product, a protection structure sleeves the part, extending out of the mounting fixed seat 15, of the interface module 33, and secondary injection molding of a coating shell 20 is carried out outside the primary product to obtain a product of second injection molding;
S500: the protection structure on the product of second injection molding is detached, and the coating shell 20 is trimmed to obtain the laryngoscope.

In S100, molds for injection molding of the first shell 11 and the second shell 12 are independent molds, whereby the first shell 11 and the second shell 12 may be independently formed; while the appearance of a mold for secondary injection molding is designed based on the appearance of the laryngoscope. In addition, in the present disclosure, during secondary injection molding, the whole laryngoscope may be fixed in the mold for secondary injection molding by the corresponding protection structures on the mounting clamping hole 14 and the mounting fixed seat 15. For the processing method of the disposable blade laryngoscope, the operation is simple, the assembly is convenient, and the sealing performance of a finished product is good.

In some possible implementation modes, positioning bars 18 are formed at one ends, with the mounting clamping hole 14, of the first shell 11 and the second shell 12 obtained by primary injection molding in S100 by injection molding; and after the first shell 11 and the second shell 12 are buckled to form the blade 10, the positioning bars 18 on the first shell 11 and the second shell 12 form part of the structure of the mounting clamping hole 14. In order to facilitate injection molding, the first shell 11 and the second shell 12 may adopt the same design, the two are buckled to form a complete blade 10, and in such a case, the blade 10 is in symmetric design.

In some possible implementation modes, in S400, the coating shell 20 of secondary injection molding outside the primary product has a tongue depressor 21 and a hollow handle 22, the protection structure coating the part, extending out of the mounting fixed seat 15, of the interface module 33 forms a filler in the injection molding process of the handle 22, and the production structure plugged in the mounting clamping hole 14 forms a filler of a visible hole 23 in the coating shell 20. With adoption of secondary forming of the tongue depressor 21 and the hollow handle 22, the processing difficulty of the forming molds of the first shell 11 and the second shell 12 may be reduced to a certain degree, which is beneficial for demolding of the molds.

In some possible implementation modes, in order to avoid the influence of a high-temperature molten material on the light source module 31, the camera module 32 and the interface module 33 in the blade 10 during the secondary injection molding process, in S200, the light source module 31, the camera module 32 and the interface module 33 are coated with thermal insulation films and installed in the mounting channel 13 together with the thermal insulation films. Of course, in the present disclosure, the first shell 11 and the second shell 12 are also made of a thermal insulation material, meanwhile, the coating shell 20 may be made of silica gel or a PP material, and the melting point of PP is relatively low, whereby the influence of high temperature on the light source module 31, the camera module 32 and the interface module 33 during the production process may be avoided; and at the same time, rapid cooling is required after the secondary injection molding to reduce the influence on the light source module 31, the camera module 32 and the interface module 33.

In some possible implementation modes, S200 further includes that when the light source module 31 and the camera module 32 are installed in the mounting clamping hole 14, a transparent window plate 17 needs to be installed at the outward end of the mounting clamping hole 14, and the protection structure may plug the mounting clamping hole 14 and protect the transparent window plate 17.

The implementation modes described above are only preferred implementation modes of the present disclosure and are not intended to limit the scope of protection of the present disclosure, and any insubstantial changes and substitutions made by those skilled in the art on the basis of the present disclosure shall fall within the scope of protection of the present disclosure.

1. A disposable blade laryngoscope, comprising:
a blade, comprising a first shell and a second shell, wherein the first shell and the second shell are buckled to form a mounting channel, one end of the mounting channel has a mounting clamping hole, and an other end forms a mounting fixed seat;
a coating shell, wherein the coating shell coats outside of the blade in an injection molding mode, an end, close to the mounting clamping hole, of the coating shell extends outwards to form a tongue depressor, the an other end, close to the mounting fixed seat, of the coating shell forms a hollow handle, and the coating shell is provided with a visible hole communicating with the mounting clamping hole; and
an optical lens assembly installed in the mounting channel, wherein an input end of the optical lens assembly is clamped in the mounting clamping hole and penetrates through the coating shell, and an output end of the optical lens assembly is installed on the mounting fixed seat and placed in the handle,
wherein the optical lens assembly comprises a light source module, a camera module and an interface module, the light source module and the camera module are installed in the mounting clamping hole, the light source module and the camera module are connected with the interface module through data lines, the interface module is installed in the mounting fixed seat, and one end of the interface module extends outwards, is placed in the handle and forms a quick insert joint,
wherein the transparent window plate blocks the mounting clamping hole.

2. (canceled)

3. The disposable blade laryngoscope according to claim 1, wherein clamping grooves are formed on inner sides of the ends, close to the mounting clamping hole, of the first shell and the second shell, a transparent window plate is clamped in the clamping groove.

4. The disposable blade laryngoscope according to claim 1, wherein a plurality of positioning bars are disposed in the first shell and the second shell, and the positioning bars arranged on the first shell and the second shell are matched with each other to clamp an outer wall of the camera module.

5. The disposable blade laryngoscope according to claim 1, wherein the first shell and the second shell are connected by ultrasonic welding.
